(19) United States
(12) Patent Application Publication
(10) Pub. No.: US 2004/0087514 A1
(43) Pub. Date: May 6, 2004

(54) Nutritional compositions

(76) Inventors: Thomas E. Hughes, Concord, MA (US); Margriet S. Westerterp-Plantenga, Maastricht (NL)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/655,406

(22) Filed: Sep. 4, 2003

Related U.S. Application Data
(60) Provisional application No. 60/408,744, filed on Sep. 6, 2002.

(51) Int. Cl. A61K 38/16; A61K 31/70; A61K 31/736

(52) U.S. Cl. 514/23; 514/2; 514/54

(57) ABSTRACT

Compositions comprising soluble fiber are effective in triggering the secretion of GLP-1. Compositions may be used in the treatment or prevention of metabolic syndrome, diabetes or obesity, the amelioration of symptoms and conditions related to metabolic syndrome, diabetes mellitus or obesity, the promotion of satiety, weight loss or maintenance of desired body weight.

This application claims the benefit of U.S. Provisional Application No. 60/408,744, filed Sep. 6, 2002.

The present invention concerns compositions comprising soluble fiber and their use in the treatment, prevention, amelioration, or control of metabolic syndrome, diabetes, or obesity and/or in the promotion of satiety, weight loss or maintenance of desired body weight.

Metabolic syndrome may be characterized by at least one of the following abnormalities: insulin resistance, hyperinsulinemia, impaired glucose tolerance, hyperlipidemia, hypercholesterolemia, hypertension, and abdominal obesity.

Obesity and diabetes are among the fastest growing segments of unmet medical needs in the developed world. Long term obesity is associated with very serious health consequences. For example, subjects with obesity particularly have an increased health risk for chronic diseases such as heart disease, type 2 diabetes, high blood pressure, stroke, and some forms of cancer. Diabetes is associated with long-term complications that affect almost every part of the body. The disease often leads to blindness, heart and blood vessel disease, kidney failure, stroke, amputation, and nerve damage. Uncontrolled diabetes can complicate pregnancy.

In order to avoid the personal distress caused by metabolic syndrome, diabetes and obesity, and also to limit the burden on medical service providers, there is an urgent need for treatments capable of controlling, e.g. treating or preventing or ameliorating or controlling, the symptoms and conditions associated with these disorders. Currently, there is a shortage of effective drug treatments. Moreover, effective nutritional approaches are highly sought after.

Glucagon-like peptide-1 (“GLP-1”) is a thirty amino acid peptide hormone, secreted from L-cells in the intestinal mucosa after ingestion of glucose, fat or mixed meals in humans. It has been shown that GLP-1 potentiates glucose-related insulin secretion at the level of pancreatic b-cells. Insulin secretion is stimulated by elevated GLP-1 blood levels only in the presence of elevated blood glucose levels. GLP-1 release further results in delayed glucose absorption and reduced glucose output from the liver, all of which help to control blood glucose. In normal weight subjects intravenous infusions of supra-physiological amounts of GLP-1 significantly accelerated satiety sensations and reduced energy intake.

The present inventors have now found a new, effective and economical way reaping the benefit of endogenously enhanced GLP-1 concentrations on hunger, satiety and glycemia.

Surprisingly, present inventors have found that (1) non-insulin stimulating nutrients, specifically non-glucose carbohydrates, e.g. galactose, xylose, fructose or mannose, preferably galactose, in combination with dietary, e.g. soluble, fiber, e.g. guar gum, beta-glucan, pectin, xanthan gum or psyllium, preferably guar gum, or (2) a mixture of dietary, e.g. soluble, fibers, preferably pectin and at least one soluble fiber chosen from e.g. guar gum, beta-glucan, pectin, xanthan gum or psyllium, preferably beta-glucan, significantly increase GLP-1 plasma levels above what is observed with a control diet. Insulin release and satiety were quantitatively related to GLP-1 release.

Compositions comprising a non-insulin stimulating nutrient, e.g. galactose, in combination with a dietary fiber, e.g. guar gum, or a mixture of dietary fibers, e.g. a mixture of beta-glucan and pectin, e.g. in the form of dietary means, supplements, nutritional or pharmaceutical formulations, hereinafter referred to as compositions of the invention, may be useful for controlling metabolic syndrome, diabetes, obesity or symptoms and conditions related to these disorders. The compositions may be self-administered for extensive periods without risk of adverse side-effects, yet are extremely effective in reducing the risk of long-term complications to the body caused by these and associated disorders.

“Symptoms and conditions related to or associated with these disorders” as defined in this application include, but are not limited to, hyperglycemia, hyperinsulinaemia, hyperlipidaemia, insulin resistance, impaired glucose metabolism, obesity, diabetic retinopathy, macular degeneration, cataracts, diabetic nephropathy, glomerulosclerosis, diabetic neuropathy, erectile dysfunction, premenstrual syndrome, vascular restenosis and/or ulcerative colitis, coronary heart disease, hypertension, angina pectoris, myocardial infarction, stroke, skin and/or connective tissue disorders, foot ulcerations, metabolic acidosis, arthritis, osteoporosis and conditions of impaired glucose tolerance.

The term “controlling” as used herein refers to the treatment, prevention, amelioration, control, delay of progression or dietary management of the diseases, symptoms, conditions and disorders described herein.

In one aspect of the present invention there is provided a use of a composition comprising a non-glucose carbohydrate and soluble fiber or a mixture of pectin and soluble fiber, in the manufacture of a medicament to trigger the secretion of GLP-1.

In a further aspect of the present invention there is provided a method for triggering GLP-1 secretion, for controlling metabolic syndrome, diabetes, obesity, or symptoms and conditions associated with these disorders, comprising administering to a subject in need of such treatment an effective amount of a composition comprising non-glucose carbohydrates and soluble fiber or a mixture of pectin and soluble fiber.

In yet a further aspect of the present invention there is provided a use of a composition comprising galactose and guar gum in the promotion of satiety or weight loss or in the maintenance of body weight.

The invention further provides a method of improving the bodily appearance of a mammal, e.g. a human, which comprises orally administering to said mammal a composition comprising non-glucose carbohydrates and soluble fiber or a mixture of pectin and soluble fiber, in a dosing effective to influence the glucose metabolism, and repeating said dosing until a cosmetically beneficial loss of body weight has occurred.

In a further aspect there is provided a use of a composition of the invention in the dietary management of metabolic syndrome, diabetes, obesity or conditions and symptoms associated with these disorders.

Suitable non-glucose carbohydrates for use according to the present invention are for example galactose, xylose, fructose or mannose. According to the invention, preferably galactose may be used.

Galactose, xylose, fructose or mannose are monosaccharides, as known and commercially available from e.g. E. Merck AG (Switzerland) or Riedel de Haen AG (Germany).

Suitable soluble fibers for use according to the present invention include, but are not limited to, agar, alginates, carubin, pectin, beta-glucan, such as oat beta-glucan, carrageenans, furcellaran, inulin, arabinogalactan, pectin and its derivatives, cellulose and its derivatives, scleroglucan, psyllium, such as psyllium seed husk, mucilages and gums. In the context of the present invention the term “pectin” refers preferably to pectins from fruits and vegetables, and more preferably to pectins from citrus fruits and apple. The term “carrageenans” as used herein encompasses in particular kappa, lambda and iota carrageenans. According to the invention, gums and mucilages are preferably plant exudates. In particular, the term “gum” as used herein refers to the commonly available vegetable gums and more particularly to konjac gum, xanthan gum, guar gum (guaran gum), locust bean gum, tara bean gum, gum tragacanth, arabic gum, karaya gum, gum ghatti, gellan gum and other related sterculia gum, alfalfa, clover, fenugreek, tamarind flour. Native and modified, e.g. hydrolyzed, soluble fibers may be used according to the invention. According to the invention, preferably guar gum, e.g. hydrolyzed guar gum, e.g. as known and commercially available under the tradename BENEFIBER® from Novartis Nutrition Corporation (Minneapolis, Minn.), may be used in combination with a non-glucose carbohydrate.

Guar gum is obtained from the ground endosperms of Cyamopsis tetragonolobus (Linné) Taub and consists mainly of a high molecular weight hydrocolloidal polysaccharide, composed of galactose and mannose units combined through glycoside linkages. Specifically, guar gum consists of linear chains of (1->4) β-D-mannopyranosyl units with α-D-galactopyranosyl units attached by (1->6) linkages. Guar gum is commercially available from e.g. Boehringer Mannheim GmbH (Germany) or Lyndal Chemical Co. (Dalton, Ga.).

Suitable soluble fibers to be used as mixtures according to the present invention are any of those referred to hereinabove. According to the invention, preferably a mixture of pectin and a soluble fiber chosen from at least one of the fibers described above, preferably β-glucan, may be used.

Glucanes generally are polysaccharides, e.g. cellulose, glycogen or starch, which form glucose upon hydrolysation. Beta-Glucans are polysaccharides of D-glucopyranose units linked by β-(1->3)- and β-(1->4) bonds. Mixed-linkage (1→3)(1→4)- β-D-Glucan (β-glucan) is the main endospermic cell wall polysaccharide of oats and barley, but also present, in lesser amounts, in rye and wheat. The subaleurone and subembryo layers of dehulled oat are particularly rich in beta-glucan, whereas in the inner starchy endosperm concentrations are low. Beta-Glucan can therefore be enriched in bran fractions by 3-4 times.

The term “Beta-glucan” as used herein refers to beta-glucan soluble fiber or a source of beta-glucan soluble fiber, e.g. a source chosen from at least one of oat, barley, rye or wheat.

Pectin is a purified carbohydrate product obtained from the dilute acid extract of the inner portion of the rind of citrus fruits or from apple pomace. It consists mainly of partially methoxylated polygalacturonic acids. Specifically, pectin is a polysaccharide of methoxylated poly- α-(1-4)-D-galacturonic acid and α-(1->2)-L-rhamnosyl-alpha-(1->4)-D-galacturonosyl residues with L-arabinose and D-galactose, D-xylose, L-fucose and D-glucuronic acid residues. It is commercially available from Riedel de Haen AG, Germany.

The term “pectin” as used herein refers to pectin soluble fiber or a source of pectin soluble fiber.

The term “soluble fiber” as used herein refers to non-starch polysaccharides characterized as being water soluble fibers, e.g. water soluble at room temperature.

Details of the active ingredients of the compositions of the invention are described in Fiedler, H. P., Lexikon der Hilfsstoffe fur Pharmazie, Kosmetik und angrenzende Gebiete, Editio Cantor Verlag Aulendorf, Aulendorf, 4th revised and expanded edition (1996); Handbook of Pharmaceutical Excipients, 3^{rd} Edition, Editors A. Wade and P. J. Weller (1994), Joint publication of American Pharmaceutical Association, Washington, USA and The Pharmaceutical Press, London, England; Remington: The Science and Practice of Pharmacy, 19^{th} Edition, 1995, Mack Publishing Company, Pennsylvania, USA; or may be obtained from the relevant manufacturers, the contents of all of which are hereby incorporated by reference.

The relative proportion of the active ingredients of the compositions of the invention will, of course, vary considerably depending on the particular type of composition concerned, e.g. whether it is a liquid or solid form, or whether it is provided in nutritional form. All indicated proportions and relative weight ranges described below are accordingly to be understood as being indicative of preferred or individually inventive teaching only and not limiting the invention in its broadest aspect.

The non-glucose carbohydrate, e.g. galactose, will suitably be present in the compositions of the invention, e.g. in solid form, e.g. in form of a powder, in an amount of from about 5 to about 95% by weight, e.g. 10 to about 90% by weight, preferably of from about 50 to about 90% by weight, most preferably in amount of from about 70 to about 80% by weight, e.g. in an amount of about 80% by weight, based on the total weight of the composition.

In a further aspect of the invention the compositions of the invention, e.g. in ready-for-consumption form, may comprise non-glucose carbohydrate, e.g. galactose, in an amount of about 5 to about 50%, e.g. about 20%, by weight based on the total weight of the composition.

The soluble fiber, e.g. guar gum, e.g. hydrolyzed guar gum, will suitably be present in the compositions of the invention, e.g. in solid form, e.g. in powder form, in an amount of from about 0.1 to about 50% by weight, e.g. 1 to about 20% by weight, preferably of from about 2.5 to about 10% by weight, most preferably in amount of about 5% by weight, e.g. in an amount of about 4% by weight, based on the total weight of the composition.

In a further aspect of the invention the compositions of the invention, e.g. in ready-for-consumption form, comprise soluble fiber, e.g. guar gum, e.g. hydrolyzed guar gum, in an amount of about 0.1 to about 10%, e.g. about 1%, by weight based on the total weight of the composition.

Non-glucose carbohydrate, e.g. galactose, and soluble fiber, e.g. guar gum, will generally be present in a ratio of about 100 to about 0.1, suitably in a ratio of about 50 to about 0.5, more suitably in a ratio of about 25 to about 1, e.g. about 20 to about 1.

Soluble fibers, e.g. beta glucan and/or pectin, will suitably be present in the compositions of the invention, e.g. in solid form, e.g. powder form, in an amount of from about 5 to about 95%, e.g. 10 to about 90% by weight, preferably of from about 50 to about 90% by weight, most preferably in amount of from about 70 to about 80% by weight, e.g. in an amount of about 80% by weight, based on the total weight of the composition.

In a further aspect of the invention the compositions of the invention, e.g. in ready-for-consumption form, comprise a mixture of soluble fibers comprising each soluble fiber, e.g. beta-glucan and pectin, in an amount of about 0.1 to about 10% by weight based on the total weight of the composition.

In one aspect of the present invention, pectin and beta-glucan will generally be present in a ratio of about 20 to about 0.05, suitably about 10 to about 0.1, more suitably about 5 to about 0.5, e.g. about 2 to about 1.

In a further aspect the present invention provides a composition comprising

(a) a non-glucose carbohydrate, preferably galactose, and soluble fiber, preferably guar gum, in a ratio of about 100 to about 0.1, e.g. in a ratio of about 100 to about 1, or

(b) a mixture of beta glucan and pectin in a ratio of about 20 to about 0.05.

In yet a further aspect of the present invention there is provided a composition in form of a dietary supplement or nutritional or pharmaceutical formulation comprising

(a) a non-glucose carbohydrate, preferably galactose, and soluble fiber, preferably guar gum, in a ratio of about 100 to about 0.1, e.g. in a ratio of about 100 to about 1, or

(b) a mixture of beta glucan and pectin in a ratio of about 20 to about 0.05, in combination with a pharmaceutically or nutritionally acceptable carrier.

In yet a further aspect of the present invention there is provided a use of a composition comprising

(a) a non-glucose carbohydrate, preferably galactose, and soluble fiber, preferably guar gum, in a ratio of about 100 to about 0.1, e.g. in a ratio of about 100 to about 1, or

(b) a mixture of beta glucan and pectin in a ratio of about 20 to about 0.05, in the manufacture of a dietary supplement or nutritional or pharmaceutical formulation for controlling metabolic syndrome, diabetes, obesity, or symptoms and conditions related to these disorders, for the promotion of satiety, weight loss or maintenance of desired body weight.

The compositions of the invention may be in medical food or beverage product form, e.g. in form of a powder for dissolution. The powder may be combined with a liquid, e.g. water, or other liquid, such as milk or fruit juice, e.g. in a ratio of powder to liquid of about 1 to about 5, to obtain a ready-to-consume composition, e.g. ready-to-drink composition or instant drink. For example, ready-to-consume drinks may contain the non-glucose carbohydrate, e.g. galactose, in an amount of from about 5 to about 50% by weight, preferably from about 10 to about 30% by weight, most preferably of from about 15 to about 20% by weight, e.g. in an amount of about 20% by weight, based on the weight of the ready-to-consume drink.

By the term “trigger secretion of GLP-1” as used herein is meant a significant, e.g. significant at p<0.05, increase in GLP-1 plasma levels above what is observed with a control diet, e.g. a diet comprising water or cellulose.

The method of treatment or use as claimed herein is applicable to normal weight, overweight and obese subjects in general. The term “overweight subjects” as used herein refers to subjects with a Body Mass Index (BMI) of 25 to 29.9 kg/m², implying an excess amount of body weight that includes muscle, bone, fat, and water, in particular an excess amount of fat. The term “obese subjects” as used herein refers to subjects with a BMI of 30 kg/m² or higher and an excess amount of body fat. It is generally agreed that men with more than 25% body fat and women with more than 30% body fat are obese.

Overweight and obese subjects are most likely to benefit from the treatment method of the present invention.

Others for whom the treatments of the present invention are envisaged include subjects suffering from metabolic syndrome and/or type 2 diabetes and/or impaired glucose tolerance.

The method of treatment according to the invention may be combined with exercise, behavior modification, and sometimes weight-loss drugs, such as amphetamines, fenfluramine, phenylpropanolamine, or mazindol, or diabetes medicines, such as sulfonylureas, biguanides, alpha-glucosidase inhibitors, thiazolidinediones, meglitinides, dipeptidyl peptidase IV (DPP IV) inhibitors, or D-phenylalanine.

According to the present invention, the compositions of the invention may be provided in form of dietary means, e.g. supplements, or in the form of a nutritional formulation, e.g. a medical food or beverage product, e.g. in form of a complete meal, part of a meal, as food additive or as powder for dissolution, or in the form of a pharmaceutical formulation, e.g. in form of a tablet, pill, sachet or capsule.

The compositions of the invention in form of dietary means, e.g. supplements, or pharmaceutical formulations may consist exclusively of non-glucose carbohydrate, e.g. galactose, and soluble fiber, e.g. guar gum, or a mixture of soluble fibers, e.g. beta-glucan and pectin, and optionally pharmaceutically acceptable carriers. Alternatively, they may be in form of nutritional formulations, e.g. medical food or beverage product, comprising other nutritional components in addition to non-glucose carbohydrate, e.g. galactose, and soluble fiber, e.g. guar gum, or in addition to the mixture of soluble fibers, e.g. beta-glucan and pectin. Furthermore, there may be provided a combined pharmaceutical formulation for simultaneous, separate or sequential use for controlling metabolic syndrome, obesity, diabetes or symptoms and conditions associated with these disorders, comprising non-glucose carbohydrate, e.g. galactose, and soluble fiber, e.g. guar gum, or a mixture of soluble fibers, e.g. beta-glucan and pectin, and one or more weight-loss drug(s) or one or more diabetes medicine(s).

In one embodiment, the compositions of the invention consist exclusively of non-glucose carbohydrate, e.g. galactose, and soluble fiber, e.g. guar gum, or a mixture of soluble fibers, e.g. beta-glucan and pectin, and optionally pharmaceutically acceptable carriers.

Compositions in accordance with the present invention may be employed for administration in any appropriate manner, e.g. enterally, e.g. orally, for example in liquid form or in solid form, preferably in liquid form. Optionally, the compositions may be administered in the form of a tube feeding solution.

Compositions for oral administration are, for example, those in single dose unit forms, such as dragées, tablets, capsules, e.g. soft gel, or sachets. Compositions may further be provided in the form of syrups, liquid suspensions, emulsions and solutions in convenient dosage forms. They are prepared in a manner known per se, for example by means of conventional mixing, granulating, confectioning, dissolving or lyophilizing processes.

For example, compositions for oral administration may be obtained by combining the active ingredients with solid carriers, optionally granulating a resulting mixture and processing the mixture or granules, if desired or necessary after the addition of suitable excipients, to form tablets or dragee cores.

Suitable physiologically acceptable carriers may be especially fillers, such as sugars, for example lactose, mannitol or sorbitol, cellulose preparations and/or calcium phosphates, for example tricalcium phosphate or calcium hydrogen phosphate, and also binders, such as starch pastes using, for example, corn, wheat, rice or potato starch, gelatin, tragacanth, methylcellulose and/or polyvinylpyrrolidone, and, if desired, disintegrators, such as the above-mentioned starches, and also carboxymethyl starch, cross-linked polyvinylpyrrolidone, agar, or alginic acid or a salt thereof, such as sodium alginate. Further excipients may be especially flow-conditioners and lubricants, for example silicic acid, talc, stearic acid or salts thereof, such as magnesium or calcium stearate, and/or polyethylene glycol. Dragée cores are provided with suitable coatings, there being used inter alia concentrated sugar solutions which may contain gum arabic, talc, polyvinylpyrrolidone, polyethylene glycol and/or titanium dioxide, or coating solutions in suitable organic solvents or solvent mixtures. Dyes or pigments may be added to the tablets or dragee coatings, for example for identification purposes or to indicate different doses of active ingredient.

Other orally administrable compositions may be in the form of hard gelatin capsules or soft, sealed capsules consisting of gelatin and a plasticizer, such as glycerol or sorbitol. The hard gelatin capsules may comprise the composition of the invention in the form of granules, for example in admixture with fillers, such as lactose, binders, such as starches, and/or glidants, such as talc or magnesium stearate, and, if desired, stabilizers. In soft capsules the composition of the invention is preferably dissolved or suspended in suitable liquids, such as fatty oils, paraffin oil or liquid polyethylene glycols, it is likewise being possible to add stabilizers.

Alternatively, the composition of the invention may be provided as a nutritional formulation, e.g. medical food, e.g. in form of a tube feeding, or in oral nutritional form as a complete meal, as part of a meal, as food additive, as a powder for dissolution, e.g. health drinks, as a solution, as a ready-made drink, optionally low calorie such as a soft drink, including juices, milk-shake, yogurt drink, smoothie or soy-based drink, in a bar, or dispersed in foods of any sort, such as baked products, cereal bars, dairy bars, snack-foods, soups, breakfast cereals, müesli, candies, tabs, cookies, biscuits, crackers, such as a rice crackers, and dairy products.

Optionally, the compositions according to the invention may be nutritionally complete, i.e. may include vitamins, minerals, trace elements as well as nitrogen, carbohydrate and fatty acid sources so that they may be used as the sole source of nutrition supplying essentially all the required daily amounts of vitamins, minerals, carbohydrates, fatty acids, proteins and the like. Accordingly, the compositions of the invention may be provided in the form of a nutritionally balanced complete meal, e.g. suited for oral or tube feeding. Preferably the compositions of the invention are for oral administration.

In one aspect the present invention provides a composition of the invention further comprising one or more viscosity lowering protein(s) chosen from at least one of wheat protein, egg protein, collagen, whey protein, casein, soy protein, pea protein, muscle protein, gluten, fibrillar protein, silk protein, or hydrolysates thereof. Compositions comprising viscosity lowering protein are disclosed in the patent application PCT/EP03/06194, in particular in the claims and the examples, the subject-matter of the disclosure is hereby incorporated into the present application by reference to this publication.

Preferably the compositions of the invention may be administered as a nutritional formulation, e.g. as part of a meal, e.g. in the form of a health drink.

It may be desirable to provide the compositions of the invention in the form of a low calorie meal replacement or other nutritional product. In this case the meal replacement or other nutritional product is preferably low fat, i.e. less than about 10 en %, or substantially fat-free, i.e. less than about 2.5 en % contributed by fat, such as about 2 en % fat, based on the total caloric content of the composition. Suitably, a single serving of a low calorie meal replacement will have a caloric value of less than about 1000 kcal (4.2 MJ), and preferably between about 200 kcal (0.8 MJ) and about 500 kcal (2.1 MJ). Suitable low calorie nutritional product may include soft drink, such as juice, smoothie or soy-based drink, or dispersed in foods of any sort, such as, dairy bars, soups, breakfast cereals, muesli, candies, tabs, cookies, biscuits, crackers, such as a rice crackers, and dairy products, such as milk-shake, yogurt drink.

Conventional additives may be included in the compositions of the invention, including any of those selected from preservatives, chelating agents, osmotic agents, buffers or agents for pH adjustment, effervescing agents, sweeteners, e.g. artificial sweeteners, flavoring agents, coloring agents, taste masking agents, acidulants, emulsifiers, stabilizers, thickening agents, suspending agents, dispersing or wetting agents, antioxidants, acidulants, texturizers, antifoam agents, and the like.

Compositions of the invention may be conveniently manufactured so as to contain up to 95% by weight, e.g. from about 20 to about 90% by weight, e.g. from about 50 to about 85% by weight non-glucose carbohydrate, e.g. galactose, and soluble fiber, e.g. guar gum, or mixture of soluble fiber, e.g. beta glucan and pectin, based on the total weight of the composition.

In one aspect of the invention the composition of the invention is a food comprising on a single serving basis e.g. about 50 g non-glucose carbohydrate, e.g. galactose, and e.g. about 2.5 g soluble fiber, e.g. guar gum, for example in about 250 ml of water.

In another aspect of the invention the composition of the invention is a food comprising on a single serving basis e.g. about 6.6 g pectin and e.g. about 3.4 g beta-glucan, e.g. in a pudding format, e.g. for administration three times daily, e.g. a daily dose about 20 g pectin and about 10 g beta-glucan.

In addition to the foregoing the present invention also provides a process for the production of a composition, e.g. nutritional or pharmaceutical formulation, as hereinbefore defined, which process comprises bringing the individual components thereof into intimate admixture and, when required compounding the obtained composition in a food or beverage product, for example ready-made drink, or in unit dosage form, for example filling said composition into a sachet.

Dependent on the form of application of the compositions of the invention, i.e. as complete meal, part of a meal, food additive, drink, sachet, tablet or capsule, the compositions of the invention may be taken once daily to e.g. five times daily. Preferably the unit doses are taken three times, e.g. with the main meals, e.g. without restriction to time of day. Preferably, the unit doses are taken together with, or shortly before, e.g. 15 minutes before, the main meals, e.g. in the morning, at noon, and in the evening.

The dose of non-glucose carbohydrate, e.g. galactose, and soluble fiber, e.g. guar gum, or mixture of soluble fiber, e.g. beta glucan and pectin, useful in performing the invention is not restricted but varies depending on, for example, the age of the subject. In terms of body weight, daily dosage may vary between from about 0.01 g to about 5 g/kg body weight/day, preferably from about 0.05 g to about 3 g/kg body weight/day, more preferably from about 0.1 g to about 2 g/kg body weight/day. In terms of ratio, the compositions useful in performing the invention may comprise a ratio of non-glucose carbohydrate:soluble fiber, e.g. galactose:guar gum, of about 100: about 0.1, e.g. about 100: about 1, or about 20: about 1, or a ratio of soluble fiber, e.g. beta glucan, : pectin of about 20 to about 0.05, e.g. about 2:1.

The compositions of the invention may be administered under the supervision of a medical specialist, or may be self-administered.

For treatment of metabolic syndrome, obesity or diabetes under clinical supervision it is possible to combine the nutritional approach with conventional pharmaceutical therapies such as weight-control drugs or diabetes medicines. For example, the composition of the invention may be provided in the form of a kit for separate, sequential or simultaneous administration in conjunction with weight-control drugs or diabetes medicines as defined hereinabove. The weight-control drugs or diabetes medicines may conveniently be formulated together with non-glucose carbohydrate, e.g. galactose, and soluble fiber, e.g. guar gum, or a mixture of soluble fibers, e.g. beta-glucan and pectin, in standard pharmaceutical dosage forms.

Optimally, the dietary supplement of the invention is consumed at least once a day on a regular basis until for example normal weight or a blood glucose level of 180 mg/dL or less 2 hours after meals has been resumed. When the supplement is supplied in the form of a food or beverage, a suitable serving size may be in the range 20 to 500 g, preferably 50 to 250 g. If provided in the form of a meal or in pharmaceutical form, one or several dosages of the non-glucose carbohydrate, e.g. galactose, and soluble fiber, e.g. guar gum, or a mixture of soluble fibers, e.g. beta-glucan and pectin, containing composition may be administered over a 24-hour period. Since these formulations are safe to consume, obese or overweight subjects or subjects with diabetes, can continue taking these supplements for as long as required, and preferably until normal weight or a blood glucose level of 180 mg/dL or less 2 hours after meals has been resumed.

Anyone perceived to be at risk from metabolic syndrome, overweight, obesity or diabetes or already suffering from these or associated disorders, can benefit from ingesting the compositions of the invention. By triggering GLP-1 secretion and hence insulin secretion and/or promoting satiety, the compositions of the invention have also the effect of counteracting the sequelae of heart disease, high blood pressure, stroke, some forms of cancer, blindness, blood vessel disease, kidney failure, amputations, and nerve damage. “High blood pressure” as used herein refers to a blood pressure persistently exceeding 140/90 mmHg (systolic/diastolic).

According to the invention it is possible to effectively ameliorate symptoms and conditions associated with metabolic syndrome, diabetes and obesity with natural compounds, which do not show any severe side effects. For example, as the actions of GLP-1 are glucose-dependent, the claimed improved method may be potentially beneficial in the treatment of diabetes without the side effect of hypoglycemia. Furthermore, the present methods are well-tolerated, for example without causing any discomfort or nausea, and simple to apply.

The utility of all the compositions of the present invention may be observed in standard clinical tests in, for example, indications as hereinabove described, for example using dosages in the range of about 0.01 to about 5 g/kg body weight/day, preferably from about 0.05 to about 3 g/kg body weight/day, more preferably from about 0.1 to about 2 g/kg body weight/day, or for example using a ratio of non-glucose carbohydrate:soluble fiber, e.g. galactose:guar gum, of about 100: about 0.1, e.g. about 100: about 1, or about 20: about 1, or a ratio of soluble fiber, e.g. beta glucan, : pectin of about 20 to about 0.05, e.g. about 2:1, for a mammal, e.g. adult and in standard animal models. The increased GLP-1/insulin secretion/promotion of satiety provided by the compositions may be observed in standard animal tests and in clinical trials, e.g. as described in the Examples below.

One human clinical trial may be affected as follows:

A single blind, placebo controlled, parallel study in 90 subjects may be performed using the composition of the invention, e.g. comprising galactose:guar gum, in a ratio of about 100: about 0.1, e.g. of about 20: about 1, or a ratio of beta glucan:pectin of about 20 to about 0.05, e.g. about 2:1, to study the effects on GLP-1 release, metabolic syndrome characteristics, weight loss and satiety. The following parameters may be assessed at baseline and after 3 month of treatment: GLP-1, OGT (oral glucose tolerance), glucose, insulin, C-peptide, TG, Glycerol, FFA, body weight, weight regain, composition of weight regain, attitude towards eating, appetite profile, and satiety (all subjects).

Another human clinical trial may be affected as follows:

To evaluate the efficacy of glucose control and insulin response to compositions of the invention vs control, compositions comprising galactose: guar gum in a ratio of about 100: about 0.1, e.g. of about 20: about 1, or a ratio of beta glucan : pectin of about 20 to about 0.05, e.g. about 2:1, are administered for 3 weeks to patients with type 2 diabetes. GLP-1, Glycemic and insulin response to a carbohydrate load of 75 g at day 1 and after treatment at day 21 are measured.

The invention will now be further illustrated by the following Examples.

### EXAMPLE 1

### Clinical Study to Assess the Effects of Non-Glucose Carbohydrates on GLP-1 Secretion in Healthy, Normal-Weight Subjects

Study Design

A randomized, placebo-controlled trial was performed in 20 subjects (12 women, 8 men) [body mass index (in kg/m²): 23]. Subjects received 5 times at least one week apart- a load consisting of galactose (50 g) alone or in combination with guar gum (2,5 g), solved in 250 ml water, or 250 ml water without any additives. Prior to each treatment a catheter was placed in an anticubital vein. Eating behavior and dietary restraint status of subjects were investigated using a validated Dutch translation of the Three Factor Eating Questionnaire (TFEQ). Subject characteristics are shown in Table 1. The study was approved by the Medical Ethics Committee of the Maastricht University.

TABLE 1
Basic characteristics of study subjects

| | Women (n = 12) | Men (n = 8) |
|---|---|---|
| Age (y) | 41 ± 14 | 39 ± 12 |
| Weight (kg) | 62 ± 8.8 | 76 ± 8.6 |
| Height (cm) | 167 ± 10 | 178 ± 8 |
| BMI (kg/m²) | 22 ± 2 | 24 ± 1 |
| Body fat (%) | 28.5 ± 7 | 18.7 ± 7.6 |
| TFEQ I (restraint) | 7 ± 4 | 4 ± 2 |
| TFEQ II (disinhibition) | 4 ± 3 | 4 ± 2 |
| TFEQ III (hunger) | 5 ± 4 | 3 ± 2 |

Results

GLP-1 release was significantly elevated in both nutrient conditions (galactose, galactose+guar gum) compared to water 30 minutes after ingestion of the load (p<0.05). Galactose+guar gum sustained this effect until 60 minutes after ingestion (p<0.05) (Table 2). The nutrient-loads significantly elevated insulin release after 30 minutes (p<0.05) (Table 2). C-Peptide, a better indicator of insulin secretion, was significantly increased 30 minutes and 60 minutes after drinking a nutrient-load compared to water ingestion (p<0.05) (Table 3). Satiety was significantly elevated at time points +60 (p<0.05) and +90 min (p<0.05) (Table 3). Regression analysis shows that satiety is a function of the increase in GLP-1 release at 30 min and 60 min after ingestion of any of the nutrient loads (p<0.05). 23% of the variation in satiety is explained by the increase in GLP-1 release. Plasma glucose was not elevated significantly after any treatment; the ingestion of galactose alone or in combination with guar gum increased galactose levels significantly; plasma free fatty acids decreased over time after all treatments; plasma glycerol levels decreased significantly after 60 minutes after all treatments; galactose and guar gum significantly decreased glycerol levels 120 minutes after ingestion (data not shown).

Table 2 GLP-1 levels (A) and insulin levels (B) after ingestion of galactose, or galactose with guar gum

| | GLP-1 (A) | | | Insulin (B) | | |
|---|---|---|---|---|---|---|
| Tim , | Con- | Galac- | Galactose + | Con- | Galac- | Galactose + |
| minutes | trol | tose | guar gum | trol | tose | guar gum |
| 0 | 3.1 | 3.8 | 4.0 | 6.5 | 7.3 | 6.6 |
| 30 | 3.3 | 4.6^{#} | 4.8# | 5.2 | 21# | 15# |
| 60 | 3.2 | 3.5 | 4.3# | 5.4 | 13 | 11 |
| 90 | 3.4 | 3.2 | 3.8 | 3.9 | 12 | 11 |
| 120 | 3.1 | 3.7 | 4.4 | 4.6 | 9.9 | 9.3 |
| 240 | 3.2 | 3.7 | 4.0 | 3.5 | 4.7 | 5.4 |

Mean values in pmol/l (A) or mU/l (B).
Significant at p < 0.05 using 5-factorial ANOVA with repeated measurement.

TABLE 3
C-peptide levels (A) or perception of satiety (B) after
ingestion of galactose, or galactose with guar gum

| | C-peptide (A) | | | Satiety (B) | | |
|---|---|---|---|---|---|---|
| Time, | Con- | Galac- | Galactose + | Con- | Galac- | Galactose + |
| minutes | trol | tose | guar gum | trol | tose | guar gum |
| 0 | 0.57 | 0.61 | 0.56 | 17 | 27 | 22 |
| 30 | 0.53 | 1.03^{#} | 0.88^{#} | 19 | 34# | 25^{#} |
| 60 | 0.52 | 0.89^{#} | 0.79^{#} | 15 | 33# | 23^{#} |
| 90 | — | — | — | 15 | 25# | 22^{#} |
| 120 | 0.48 | 0.88 | 0.71 | 14 | 31# | 21 |
| 240 | — | — | — | 10 | 20 | 20 |

Mean values nmol/l (A) and from 100 mm Visual Analog Scale (B).
^{#}Significant at p < 0.05 using 5-factorial ANOVA with repeated measurement

Discussion of Results

Galactose (50 g) in combination with guar gum (2,5 g) evoked sustained elevated GLP-1 release 30-60 minutes after ingestion, in comparison to water. Insulin release and satiety were quantitatively related to GLP-1 release.

### EXAMPLE 2

### Clinical Study to Assess the Effects of Non-Glucose Carbohydrates Before a Breakfast on GLP-1 Secretion in Healthy, Normal-Weight Subjects

Study Design

A randomized, placebo-controlled trial was performed in 30 subjects (15 women, 15 men) [body mass index (23 kg/m²)]. Subjects received 2 times—at least one week apart—a load consisting of galactose (50 g) with guar gum (2,5 g), solved in 250 ml water, or 250 ml water without any additives before breakfast. All subjects underwent an oral glucose tolerance test following the standards of the world health organization. Every test day, subjects arrived at the lab after an overnight fast. A catheter was placed in an anticubital vein. After the baseline blood sample was taken, subjects drank the galactose guar gum load or water. They were instructed to drink everything in between 5 minutes. 15 minutes after finishing the load, they had a standard breakfast having an energy density of 3.9 kJ/g and consisting of two slices of brown bread (100 g), baked egg (85 g) and 300 ml skim milk (1.9 MJ). The energy distribution was 48.8 energy-percent (en %) carbohydrates, 28.5 en % protein and 22.6 en % fat. Again they were instructed to fully finish the meal in between 15 minutes. 30 minutes as well as 60, 90 and 120 minutes after the load blood samples were taken. Blood samples were analysed for GLP-1, insulin, glucose and free fatty acids. Eating behavior and dietary restraint status of subjects were investigated using a validated Dutch translation of the Three Factor Eating Questionnaire (TFEQ). Subject characteristics are shown in Table 4.

The study was approved by the Medical Ethics Committee of the Maastricht University.

TABLE 4
Basic characteristics of study subjects

| | Women (n = 15) | Men (n = 15) |
|---|---|---|
| Age (y) | 34.6 ± 13.5 | 27.3 ± 11.1 |
| Weight (kg) | 65.2 ± 6.5 | 74.22 ± 3.9 |
| Height (cm) | 169 ± 1 | 182 ± 1 |
| BMI (kg/m²) | 23 ± 1.6 | 22 ± 1.3 |
| Body fat (%) | 27.1 ± 7 | 15 ± 6.8 |
| TFEQ I (restraint) | 7 ± 3.3 | 4 ± 2 |
| TFEQ II (disinhibition) | 4.1 ± 2.5 | 3.7 ± 1.4 |
| TFEQ III (hunger) | 4.5 ± 3.2 | 3.6 ± 2.4 |

Results

GLP-1 release was significantly elevated in the galactose with guar gum condition compared to water 30, 45 and 60 minutes after ingestion of the load (p<0.05) (Table 5). Compared to Example 1, GLP-1 levels were about twice as high by adding a breakfast. Insulin peak was delayed in the galactose+guar gum condition compared to the control condition. Insulin peaked and was significantly elevated at 90 minutes in the test condition (p<0.05), 30 minutes later than water, but stayed significantly elevated until the end of measurement (p<0.05) (Table 5). Satiety occurred as a function of GLP-1-level in the test condition, but not in the control condition (p<0.05). Glucose was not significantly different at 30 minutes; after 45 and 60 minutes, glucose was significantly elevated after ingestion of water and breakfast; at 90 minutes glucose release peaked and was significantly elevated in the test condition, area under the curve was significantly lower compared to the control condition (data not shown). Free fatty acids decreased continuously over time, significantly more after consumption of galactose with guar gum (data not shown).

TABLE 5
GLP-1 release (A) and insulin release (B) after ingestion of
galactose with guar gum and water (control) and a standard breakfast.

| | GLP-1 (A) | | Insulin (B) | |
|---|---|---|---|---|
| Time, | | Galactose + | | Galactose + |
| minutes | Control | guar gum | Control | guar gum |
| 0 | 4.55 ± 3.22 | 5.95 ± 9.01 | 6.52 ± 2.71 | 6.33 ± 2.65 |
| 30 | 8.41 ± 5.16 | 14.58 ± 10.41^{#} | 17.20 ± 11.29 | 20.81 ± 9.95 |
| 45 | 8.41 ± 4.17 | 13.27 ± 8.85^{#} | 36.80 ± 19.41 | 29.58 ± 25.10 |
| 60 | 6.97 ± 3.94 | 9.92 ± 9.66^{#} | 40.33 ± 22.30 | 29.43 ± 24.52 |
| 90 | 6.64 ± 3.69 | 8.12 ± 8.40 | 31.12 ± 18.59 | 38.81 ± 26.00^{#} |
| 120 | 7.56 ± 3.80 | 8.54 ± 9.33 | 24.54 ± 12.54 | 38.06 ± 19.08^{#} |

Mean values in pmol/l (A) and in mU/l (B).
^{#}Significant at p < 0.05 using 2-factorial ANOVA with repeated measurement.

Discussion of Results

Galactose (50 g) in combination with guar gum (2,5 g) consumed 15 min before a standard breakfast evoked sustained, elevated GLP-1 release 30-60 minutes after ingestion, in comparison to water consumed 15 min before breakfast. The galactose +guar gum load before breakfast increased the amplitude of GLP-1 release. Satiety was a function of GLP-1 release in the test condition with the galactose guar gum load. The ingestion of galactose with guar gum in combination with the breakfast resulted in sustained elevated insulin levels.

### EXAMPLE 3

### Clinical Study to Assess the Effects of Non-Glucose Carbohydrates Before a Breakfast on GLP-1 Secretion in Overweight Subjects

Study Design

A study was performed to investigate GLP-1 release after ingestion of a galactose (50 g,) guar gum (2,5) load (250 ml, 836 kJ) and a standard breakfast consisting of 100 g of brown bread, 85 g baked eggs and 300 ml low fat milk (1,9 MJ) in overweight subjects compared to a release after ingestion of water (250 ml) and a breakfast. The protocol was mainly as described in Example 2.

After blood sampling subjects filled in visual analogue scales for hunger and satiety. Blood samples were analysed for GLP-1, insulin, glucose and free fatty acids.

TABLE 6
Subject characteristics

| | |
|---|---|
| Height (cm) | 1.71 |
| Weight (kg) | 89.36 ± 9.09 |
| BMI | 30.46 ± 2.70 |
| Waist (cm) | 96 ± 8.11 |
| Hip (cm) | 110.48 ± 6.8 |
| WHR | .87 ± .08 |
| TFEQ (F1) | 6.84 ± 3.98 |
| TFEQ (F2) | 6.41 ± 3.03 |
| TFEQ (F3) | 5.64 ± 3.85 |

Results

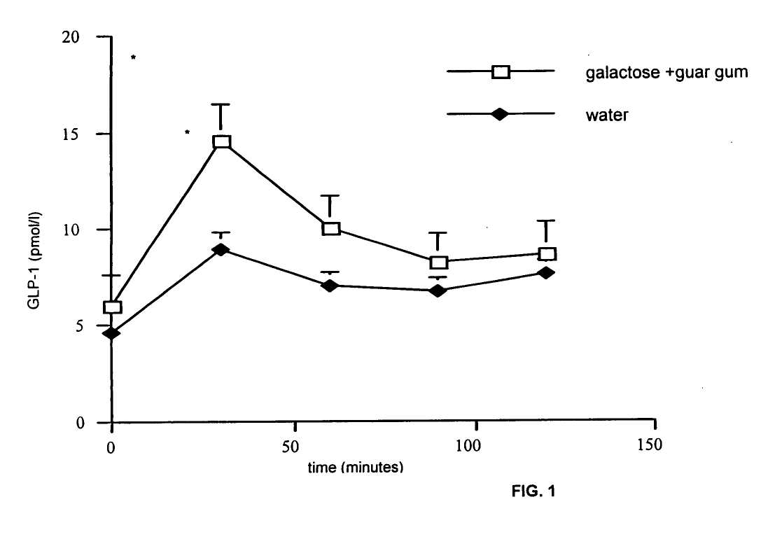

GLP-1 was significantly different between conditions (F_{1, 14}=5.67; P=0.0001). GLP-1 release appeared to be significantly increased in the galactose guar gum condition at 30 minutes (p<0.0001) and 60 minutes (p<0.0001) compared to ingestion of water before breakfast (FIG. 1). Insulin release was blunted after ingestion of the galactose guar gum load, compared to water (F_{1, 14}=4.75; P=0.0003). At 90 minutes total insulin release in not different between the two conditions.

Glucose was not different at any time between conditions, neither were free fatty acids (data not shown).

FIG. 1 shows GLP-1 release in overweight subjects.

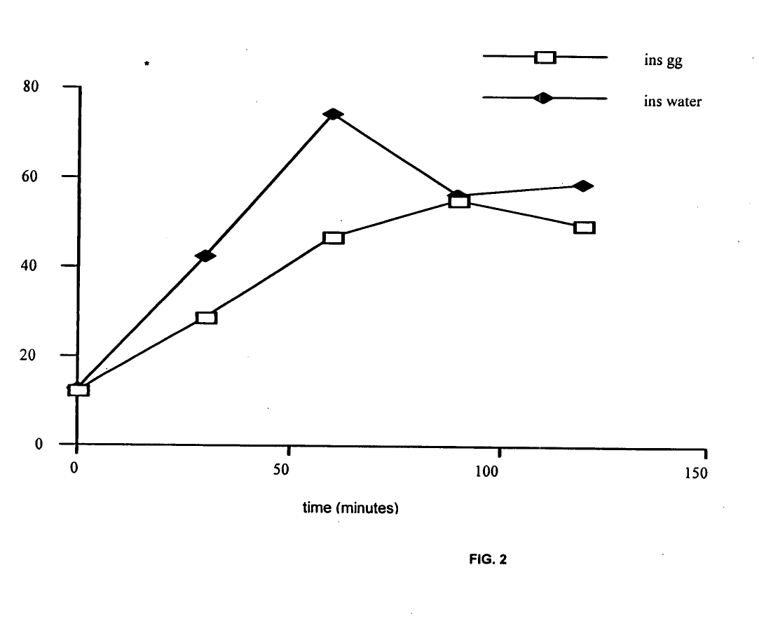

FIG. 2 shows Insulin release in overweight subjects.

### Example 4

### Clinical Study to Assess the Effects of Beta Glucan and Pectin on GLP-1 Secretion in Healthy, Normal-Weight Subjects

A placebo-controlled trial was performed in 11 subjects (7 women, 4 men) [body mass index (in kg/m²): 25.6]. Subject characteristics are shown in Table 7. Subjects consumed for 3 weeks a placebo comprising the non-fermentable fiber methyl-cellulose, and after a 1 week washout phase for further 3 weeks the active comprising the fermentable fibers beta glucan and pectin. In the first phase subjects consumed 3 times per day a supplement containing 10 g of methyl-cellulose in the form of a pudding. In the second phase subject consumed 3 times a day a supplement containing a mix of 10 g pectin/beta-glucan (ratio of 2:1) in the form of a pudding, i.e. 3 times a day 6.6 g pectin plus 3.4 g beta-glucan. After the 3 week consumption of the supplement subjects consumed a standardized liquid meal and postprandial GLP-1 response was monitored (Table 8, 9).

TABLE 7
Basic characteristics of study subjects

| | 7 Women, 4 men (n = 11) |
|---|---|
| Age (y) | 30.7 ± 7.3 |
| Weight (kg) | 75.1 ± 20.0 |
| Height (cm) | 170 ± 9 |
| BMI | 25.6 ± 0.9 |

TABLE 8
Effect of fiber supplementation on prandial GLP-1 release

| Time, | | |
|---|---|---|
| minutes | Methyl-cellulose | Beta-glucan + Pectin |
| 0 | 5.5 | 5.5 |
| 15 | 9.5 | 14 |
| 30 | 8 | 11 |
| 45 | 7.5 | 8.5 |
| 60 | 7 | 8.25 |
| 120 | 7 | 11 |
| 240 | 6 | 6 |

Mean values in pmol/l.

TABLE 9
Effect of fiber supplementation on prandial GLP-1 release

| | Phase 1 | | Phase 2 | |
|---|---|---|---|---|
| | Initial | Final | Initial | Final |
| Fasting² | 7.18 ± 7.22 | 5.45 ± 4.77 | 5.73 ± 5.31 | 5.50 ± 4.30 |
| Fed AUC | | 1723 ± 1053 | | 2221 ± 1130 |
| Excursion³ | | 5.00 ± 3.76 | | 12.77 ± 11.02 |

¹Mean ± SD
²No significant differences Initial and Final values within each Phase or between values for Phase 1 vs Phase 2
³Excursion = Maximal fed state value − Baseline. Difference between Phase 1 vs Phase 2, P < 0.03

Discussion of Results

10 g Pectin/beta-glucan (ratio of 2:1) evoked sustained elevated GLP-1 release 15-120 minutes after ingestion, in comparison to methyl-cellulose.

### EXAMPLE 5

### Preparation of a Medical Food

Medical food in form of an instant drink, 0.96 kcal/mL, fat free, orange juice like. Recipe in agreement with FSMP (Food For Special Medical Purposes) legislation for “incomplete” supplement.

The ingredients are homogeneously dispersed using a dry mixing unit and filled in one serving sachet.

The content of one serving sachet is added to a shaker containing 250 mL of cold water and properly shaked until a homogenous solution is obtained. The product is clear after approximately 5 min standing time.

| Component | Quantity/serving (g) |
|---|---|
| Galactose | 50 |
| Whey protein | 10 |
| Gum Guar | 2.5 |
| Acidity regulator (HCl) | 0.2 |
| Orange flavor | 0.2 |
| Sodium Phosphate | 0.1 |
| Potassium Chloride | 0.1 |
| Vit C | 0.025 |
| Iron sulfate | 0.02 |
| Zinc sulfate | 0.02 |
| Vitamin E | 0.003 |
| Niacinamide | 0.003 |
| Manganese sulfate | 0.003 |
| Calcium D pantothenate | 0.001 |
| Copper sulfate | 0.001 |
| Vit A | 0.001 |
| Vit B2 | 0.0003 |
| Vit B1 | 0.0003 |
| Vit B6 | 0.0003 |
| Potassium iodide | 0.0002 |
| Sodium molybdate | 0.00008 |
| Sodium selenite | 0.00004 |
| Folic acid | 0.00004 |
| Chromium chloride | 0.00004 |
| Vit K1 | 0.00003 |
| Biotin | 0.000008 |
| Vit D3 | 0.000002 |
| Vit B12 | 0.0000002 |
| TOTAL | ca: 63 g |

### EXAMPLE 6

### Preparation of a Beverage

Guar gum is mixed with galactose and added to a tank with cold water (15-25° C.) under continuous agitation. The composition is mixed for 15 minutes, then the antifoam agent, whey protein, acidity regulator and flavor are added. The composition is kept under agitation for one hour. The product is pre-heated up to 50° C. and homogenized in a two steps homogenization (200/50). The product is pasteurized at 90° C., 50s holding time, and filled in a 250 mL bottle under aseptic conditions.

| Component | Quantity (g)/250 |
|---|---|
| Water | 187.1 |
| Galactose | 50 |
| Whey protein | 10 |
| Gum guar | 2.5 |
| Acidity regulator (HCl) | 0.2 |
| Orange flavor | 0.2 |
| Antifoam (Tween ® 80 | 0.05 |

### Example 7

### Preparation of a Dietary Supplement

The different ingredients are admixed together and mixed with 90 g of water at temperature of about 25° C., under continuous agitation, for 30 minutes.

The product is pasteurized at 90° C., 50s holding time, and filled in a 250 mL bottle under aseptic conditions.

| Component | Quantity (g)/250 |
|---|---|
| Water | 225 |
| Beta-glucan | 0.2 |
| Whey protein | 7.5 |
| Pectin | 0.8 |
| Saccharose | 15 |
| Acidity regulator (citric acid) | 0.2 |
| Citrus flavor | 0.25 |
| Vitamin mineral mix | 1 |
| Antifoam (Tween ® 80) | 0.05 |

We claim:

1. A method of triggering glucagon like peptide 1 secretion in a mammal comprising the step of administering to said mammal an effective amount of a composition comprising a non-glucose carbohydrate and a soluble fiber.

2. The method of claim 1, wherein said method is for a condition related to glucose metabolism.

3. The method of claim 2, wherein said condition is selected from the group consisting of metabolic syndrome, diabetes, obesity, satiety, weight loss and weight maintenance.

4. The method of claim 1, wherein said non-glucose carbohydrate is galactose.

5. The method of claim 4, wherein said soluble fiber is guar gum.

6. The method of claim 1, wherein the ratio, by weight, of said non-glucose carbohydrate to said soluble fiber is about 100 to about 0.1.

7. The method of claim 6 wherein said ratio is from about 100 to about.

8. The method of claim 1, wherein said composition further comprises a viscosity lowering protein.

9. The method of claim 8, wherein said viscosity lowering protein is selected from the group consisting of wheat protein, egg protein, collagen, whey protein, casein, soy protein, pea protein, muscle protein, gluten, fibrillar protein, silk protein and hydrolysates thereof.

10. A method of triggering glucagon like peptide 1 secretion in a mammal comprising the step of administering to said mammal an effective amount of a composition comprising pectin and a soluble fiber.

11. The method of claim 10, wherein said method is for a condition related to glucose metabolism.

12. The method of claim 11, wherein said condition is selected from the group consisting of metabolic syndrome, diabetes, obesity, satiety, weight loss and weight maintenance.

13. The method of claim 10, wherein said soluble fiber is selected from the group consisting of guar gum, beta-glucan, xanthan gum and psyllium.

14. The method of claim 13, wherein said soluble fiber is beta-glucan.

15. The method of claim 13, wherein the ratio, by weight, of said beta-glucan to said pectin is about 20 to about 0.05 by weight.

16. The method of claim 10, wherein said composition further comprises a viscosity lowering protein.

17. The method of claim 16, wherein said viscosity lowering protein is selected from the group consisting of wheat protein, egg protein, collagen, whey protein, casein, soy protein, pea protein, muscle protein, gluten, fibrillar protein, silk protein and hydrolysates thereof.

18. A composition comprising galactose and guar gum, wherein said galactose and guar gum are present in a ratio, by weight, of about 100 to about 0.1.

19. The composition of claim 18 further comprising a viscosity lowering protein selected from the group consisting of wheat protein, egg protein, collagen, whey protein, casein, soy protein, pea protein, muscle protein, gluten, fibrillar protein, silk protein and hydrolysates thereof.

20. A composition comprising pectin and beta-glucan, wherein said pectin and beta-glucan are present in a ratio, by weight, of about 20 to about 0.05.

21. The composition of claim 19 further comprising a viscosity lowering protein selected from the group consisting of wheat protein, egg protein, collagen, whey protein, casein, soy protein, pea protein, muscle protein, gluten, fibrillar protein, silk protein and hydrolysates thereof.

22. A method of improving the bodily appearance of a mammal comprising the steps of
a) orally administering to said mammal a composition comprising a non-glucose carbohydrate and a soluble fiber in a dosing effective to influence glucose metabolism, and
b) repeating said administering until a cosmetically beneficial loss of body weight has occurred.

23. A method of improving the bodily appearance of a mammal comprising the steps of
a) orally administering to said mammal a composition comprising a pectin and a soluble fiber in a dosing effective to influence glucose metabolism, and
b) repeating said administering until a cosmetically beneficial loss of body weight has occurred.
